(19) United States
(12) Patent Application Publication
(10) Pub. No.: US 2017/0043102 A1
(43) Pub. Date: Feb. 16, 2017

(54) CANNULA ADAPTER FOR MEDICAL DEVICES

(71) Applicant: MARINA MEDICAL INSTRUMENTS, INC., Sunrise, FL (US)

(72) Inventors: BENJAMIN M. MOSES, Fort Lauderdale, FL (US); ALEXANDER BARRON, DAVIE, FL (US)

(21) Appl. No.: 15/234,623

(22) Filed: Aug. 11, 2016

Related U.S. Application Data
(60) Provisional application No. 62/203,610, filed on Aug. 11, 2015.

(51) Int. Cl. A61M 5/34; A61M 5/48; A61M 1/00

(52) U.S. Cl. CPC A61M 5/347; A61M 1/0086; A61M 5/484; A61M 2202/08

(57) ABSTRACT

A cannula adapter can include an adapter body, a breakpoint, and a coupling mechanism. The adapter body can have an interior channel with opposing openings. The first opening of the interior channel can be sized and shaped for insertion of a cannula and the second opening can be sized and shaped for a connection point of a medical device. The breakpoint can separate the adapter body into two discrete sections when an applied transverse force meets or exceeds its failure threshold, decreasing a likelihood of causing injury to a patient and the user of the medical device. The coupling mechanism can couple a bottom end of the adapter body to the connection point of the medical device, forming an air-tight connection between the cannula and the medical device that increases a failure threshold of the adapter body for withstanding applied longitudinal forces and torque.

### CROSS-REFERENCE TO RELATED APPLICATIONS

This utility application claims the benefit of U.S. Provisional Patent 62/203,610 filed on Aug. 11, 2015 titled “ENHANCEMENTS TO A SYRINGE OR LIPOSUCTION HANDLE TO CANNULA ADAPTER.” The entire contents of 62/203,610 are incorporated by reference herein in their entirety.

### BACKGROUND

The present invention relates to the field of medical adapters and, more particularly, to an improved cannula adapter for medical devices.

A variety of medical procedures utilize cannulas (small tubes) that are inserted into a patient. The cannula is often connected to another medical device, such as a syringe or a specialized wand, via an adapter. When the medical procedure involves high pressure and/or suction or pressure differentials between the patient's body and the medical device, the conventional adapters connecting to the cannula are known to fail, causing undo stress upon the physician and patient.

For example, in lipoplasty, fat is injected and/or removed from the patient's body using an inserted cannula that is connected to a syringe or the handle of the liposuction tool with an adapter. During the procedure, a large amount of pressure is generated by the medical device and propagated through the adapter to the cannula to inject or remove the fat. This large amount of pressure has been known to cause the adapter to violently separate from the cannula and/or the medical device, which can be dangerous to the patient.

The issue of adapter failure is especially critical in the case of lipoplasty and other similar cosmetic surgery procedures. The fat being injected or suctioned often becomes stuck in the cannula-adapter-tool apparatus due to the viscous nature of the fat. This exacerbates the built-up pressure, increasing the likelihood of adapter failure.

### BRIEF SUMMARY

One aspect of the present invention can include a cannula adapter comprised of an adapter body, a breakpoint, and a coupling mechanism. The adapter body can have an interior channel with opposing openings. A first opening of the interior channel can be located on a top surface of the adapter body and can be sized and shaped to match a respective size and a respective shape for insertion of a cannula. A second opening of the interior channel can be sized and shaped to match a respective size and a respective shape of a connection point of a medical device. The breakpoint can be integrated throughout a horizontal cross-section of the adapter body. Responsive to an application of transverse force to the adapter body that meets or exceeds a failure threshold of the breakpoint, the breakpoint can separate the adapter body into two discrete sections. Separation of the adapter body can decrease a likelihood of causing injury to a patient and the user of the medical device. The coupling mechanism can couple a bottom end of the adapter body to the connection point of the medical device. Engagement of the coupling mechanism can form an air-tight connection between the inserted cannula and the medical device that increases a failure threshold of the adapter body for withstanding longitudinal forces and torque applied by a user of the medical device during a medical procedure.

Another aspect of the present invention can include a system that includes a cannula, a syringe, and an adapter that creates an air-tight connection between the cannula and the syringe. The adapter can be comprised of an adapter body, a breakpoint, and a coupling mechanism. The adapter body can have an interior channel with opposing openings. A first opening of the interior channel can be located on a top surface of the adapter body and can be sized and shaped to match a respective size and a respective shape for insertion of a cannula. A second opening of the interior channel can be sized and shaped to match a respective size and a respective shape of a connection point of the syringe. The breakpoint can be integrated throughout a horizontal cross-section of the adapter body. Responsive to an application of transverse force to the adapter body that meets or exceeds a failure threshold of the breakpoint, the breakpoint can separate the adapter body into two discrete sections. Separation of the adapter body can decrease a likelihood of causing injury to a patient and the user of the medical device. The coupling mechanism can couple a bottom end of the adapter body to the connection point of the syringe. Engagement of the coupling mechanism can form an air-tight connection between the inserted cannula and the syringe that increases a failure threshold of the adapter body for withstanding longitudinal forces and torque applied by a user of the medical device during a medical procedure.

Yet another aspect of the present invention can include a system comprised of a cannula, a manually-manipulated tool for a lipoplasty apparatus having a threaded connection point, and an adapter that creates an air-tight connection between the cannula and the tool. The adapter can include an adapter body, a breakpoint, and a coupling mechanism. The adapter body can have an interior channel with opposing openings. A first opening of the interior channel can be located on a top surface of the adapter body and can be sized and shaped to match a respective size and a respective shape for insertion of a cannula. A second opening of the interior channel can be sized and shaped to match a respective size and a respective shape of a connection point of the tool. The breakpoint can be integrated throughout a horizontal cross-section of the adapter body. Responsive to an application of transverse force to the adapter body that meets or exceeds a failure threshold of the breakpoint, the breakpoint can separate the adapter body into two discrete sections. Separation of the adapter body can decrease a likelihood of causing injury to a patient and the user of the lipoplasty apparatus. The coupling mechanism can couple a bottom end of the adapter body to the connection point of the tool. Engagement of the coupling mechanism can form an air-tight connection between the inserted cannula and the tool that increases a failure threshold of the adapter body for withstanding longitudinal forces and torque applied by a user of the tool during a lipoplasty procedure.

### BRIEF DESCRIPTION OF THE SEVERAL VIEWS OF THE DRAWINGS

FIG. 1 is a block diagram of system for utilizing a break-away adapter in accordance with embodiments of the inventive arrangements disclosed herein.

FIG. 2 is a collection of views for an exemplary break-away adapter in accordance with embodiments of the inventive arrangements disclosed herein.

FIG. 2A illustrates use of the break-away cannula adapter with a Toomey syringe in accordance with embodiments of the inventive arrangements disclosed herein.

FIG. 3 is collection of views for an exemplary break-away adapter in accordance with embodiments of the inventive arrangements disclosed herein.

FIG. 3A illustrates use of the break-away cannula adapter with a Luer Lok syringe in accordance with embodiments of the inventive arrangements disclosed herein.

### DETAILED DESCRIPTION

The present invention discloses an improved adapter for connecting a cannula to a medical device. The adapter can utilize a coupling mechanism that creates an air-tight connection between the cannula and the medical device, which increases the adapter's ability to withstand the longitudinal forces and torque that are commonly applied during medical procedures. The adapter can also include a breakpoint that separates the body of the adapter when transverse forces become too great, mitigating injury to the patient and the use of the medical device.

As will be appreciated by one skilled in the art, aspects of the present invention may be embodied as a system or method. Aspects of the present invention are described below with reference to flowchart illustrations and/or block diagrams of methods and apparatus (systems) according to embodiments of the invention.

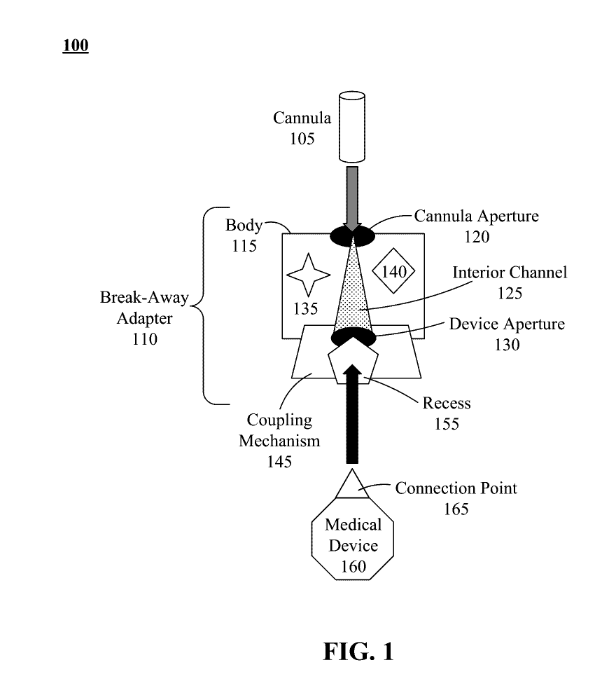

FIG. 1 is a block diagram of system 100 for utilizing a break-away adapter 110 in accordance with embodiments of the inventive arrangements disclosed herein. As is known in the Art, adapters are used to connect cannulas 105 to a variety of medical devices 160 for various medical procedures. Conventional adapters can be prone to failure due to the stresses applied by manual manipulation, such as in the case of lipoplasty procedures, where a lipoplasty tool must be vigorously manipulated.

The break-away adapter 110 of system 100 can be superior to conventional adapters as its design can create an air-tight connection between the cannula 105 and a medical device 160, which increases the break-away adapter's 110 ability to withstand longitudinal forces and torque, and can include a breakpoint 135 that separates the body 115 of the break-away adapter 110 when transverse forces become too great, mitigating injury to the patient and the use of the medical device 160.

The cannula 105 can conform to the standards known in the Art. The medical device 160 can represent a variety of medical items commonly used with cannula 105 and/or break-away adapter 110. Common examples of the medical device 160 can include, but are not limited to, syringes, manually-manipulated tools of larger medical apparatuses (e.g., a wand or scope), and the like.

The break-away adapter 110 can be comprised of a body 115 and a coupling mechanism 145. The body 115 and/or coupling mechanism 145 can be made of materials that are appropriate for use in medical procedures. The body 115 can have a longitudinal interior channel 125 that provides passage of materials between the cannula 105 and the medical device 160.

The interior channel 125 can have a cannula aperture 120 where the cannula 105 can be inserted and a device aperture 130 where a connection point 165 of the medical device 160 can be inserted. Both apertures 120 and 130 can be sized and shaped for their respective inserted elements.

The body 115 can further include a breakpoint 135 and a pressure release mechanism 140. The breakpoint 135 can be a mechanism embedded within the body 115 that automatically separates the body 115 when excessive transverse forces are applied to the break-away adapter 110. This separation can be a controlled, safe event, not a catastrophic event, for mitigating damage and/or injury to the patient and/or user of the medical device 160.

For example, during a liposuction procedure, if a patient jerks suddenly while a physician is using a liposuction syringe 160 that uses a break-away adapter 110, the breakpoint 135 can allow the syringe 160 to be easily separated.

The pressure release mechanism 140 can be a mechanism to decrease pressure gradients that build-up between the medical device 160, break-away adapter 110, and/or cannula 105 during a medical procedure. The pressure release mechanism 140 can conform to traditional (e.g., release dial valve of rubber inflation bulbs) and/or proprietary mechanisms. The pressure release mechanism 140 can, therefore, provide additional assistance in mitigating failures of the break-away adapter 110 during the medical procedure.

The coupling mechanism 145 can couple the body 115 of the break-away adapter 110 to the connection point 165 of the medical device 160. The specific implementation of the coupling mechanism 145 can vary based upon the requirements of the connection point 165 of the medical device 160.

For example, the coupling mechanism 145 can be a set of cutting threads that physically cuts into the plastic nose of a syringe. As another example, the coupling mechanism 145 can be a nut-bolt mechanism where the body 115 of the break-away adapter 110 and the connection point 165 both include threads that the nut screws onto.

The coupling mechanism 145 can include a recess 155 for accommodating the connection point 165 and the medical device 160. The recess 155 can sized and shaped to rest upon the connection point 165 and/or medical device 160 to provide an air-tight seal between the medical device 160 and coupling mechanism 145.

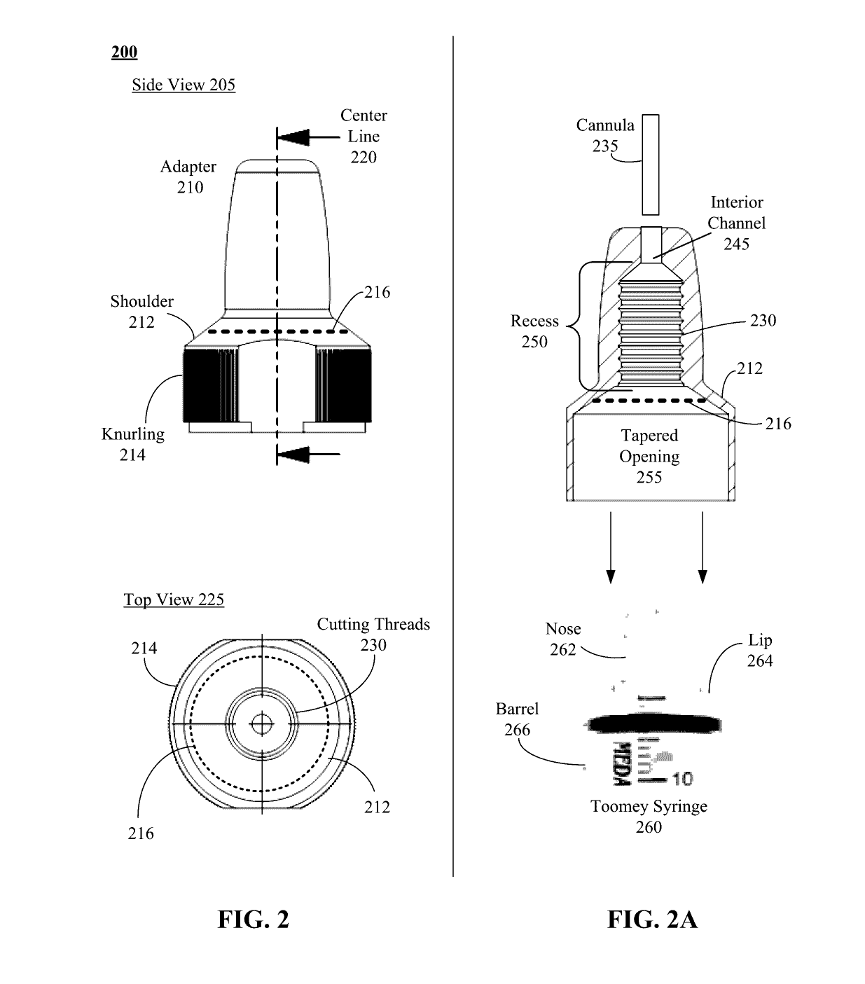

FIG. 2 is a collection 200 of views 205 and 225 for an exemplary break-away adapter 210 in accordance with embodiments of the inventive arrangements disclosed herein. Collection 200 can depict a side view 105 and a top view 225 of the break-away adapter 210.

As shown in the side and the top views 205 and 225, the adapter 210 can be symmetrically cylindrical around a center line 220. It should be appreciated that the size and shape of the adapter 210 can conform to traditional industry or proprietary tolerances (e.g., +/−0.0001 mm). In this embodiment, the adapter 210 can include a shoulder 212, knurling 214, a breakpoint 216, cutting threads 230, and the like.

Knurling 214 can provide greater grip traction for manipulating the adapter 210 during the medical procedure. The breakpoint 216, as shown here, can be located on the shoulder 212 of the adapter 210; other positions of the breakpoint 216 can also be contemplated.

This embodiment of the adapter 210 can be suited for coupling the cannula 235 with a Toomey syringe 260, as shown in FIG. 2A, or a liposuction handle (not shown). As depicted in FIG. 2A, the Toomey syringe 260 can be inserted into a tapered opening 255 of the adapter 210. The adapter 210 shoulder 212 can fit against the lip 264 and/or the barrel 266 of the syringe 260, placing the nose 262 into the recess 250 of the adapter 210.

It should be appreciated that the direct contact between the adapter 210 and the syringe 260 can provide the adapter 210 with the ability to better withstand the longitudinal forces and torque that are often applied during medical procedures without failure. That is, conventional adapters that do not have such a tight coupling to the syringe 260 can fail under lower amounts of longitudinal and rotational stresses than the break-away adapter 210.

For example, in lipoplasty procedures, differences in pressure gradients from harvesting the fat cells and injecting the fat cells can occur. That is, due to the viscus nature of fat, significant longitudinal forces must be applied to the composite apparatus of the syringe 260, adapter 210, and cannula 235 to inject fat into subcutaneous regions of the human body. This adapter 210 can be well suited to accommodate these longitudinal forces, which are known to overwhelm conventional adapters.

Rotating the syringe 260 can couple the syringe 260 to the adapter 210 via the cutting threads 230 within the recess 250. Rotation of the syringe 260 can cause the cutting threads 230 to cut threads into the nose 162 of the syringe 260 (i.e., the coupling mechanism), creating an air-tight seal that connects to the cannula 235 via the interior channel 245. Thus, the adapter 210 can be securely attached to a Toomey syringe 260 or a liposuction handle using a mechanism that provides external fixation to the outer wall of the syringe or liposuction handle, and, by extension, to any other apparatus connected to the syringe 260.

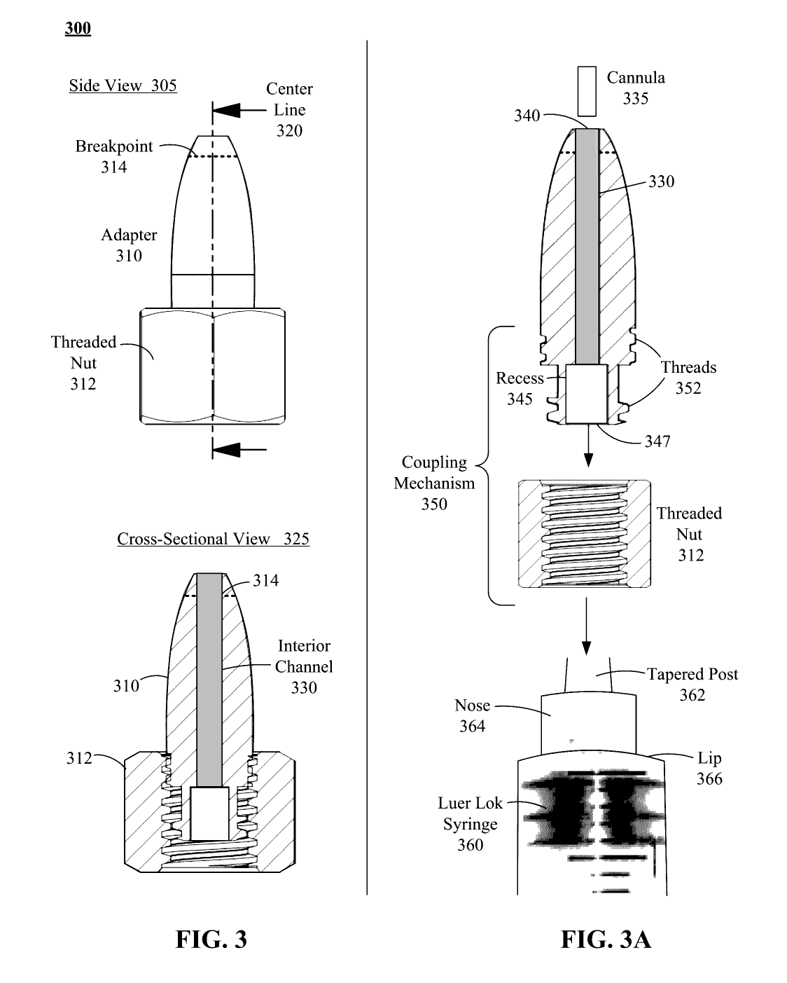

FIG. 3 is a collection 300 of views 305 and 325 for an exemplary break-away adapter 310 in accordance with embodiments of the inventive arrangements disclosed herein. Collection 300 can depict a side view 305 and a cross-sectional view 325 of the break-away adapter 310.

As shown in the side and the cross-sectional views 305 and 325, the adapter 310 can be symmetrically cylindrical around a center line 320. It should be appreciated that the size and shape of the adapter 310 can conform to traditional industry or proprietary tolerances (e.g., +/−0.0001 mm). The breakpoint 314, as shown here, can be located near the top end of the adapter 210, near where the cannula 335 is inserted; other positions of the breakpoint 314 can also be contemplated.

This embodiment of the adapter 310 can utilize a coupling mechanism 350 comprised of threads 352 on the adapter 310 and a threaded nut 312 to couple the cannula 335 with a Luer Lok syringe 360, as shown in FIG. 3A, or a liposuction handle (not shown). As own in FIG. 3A, the adapter 310 can have threads 352 on it bottom end where the threaded nut 312 it to be coupled. The threaded nut 312 can then be fastened against the nose 264 of the Luer Lok syringe 360

In one embodiment, the threaded nut 312 can couple with inner threads within the nose 364 of the Luer Lok syringe 360, connecting the interior cavity of the syringe 360 to the cannula 335 via the interior channel 330 of the adapter 310. In another embodiment, the threaded nut 312 can be fastened against the outer surface of nose 364 Luer Lok syringe 360 via cutting threads (not shown) on the inside of the threaded nut 312, which cut threads into the nose 364. In yet another contemplated embodiment, the threaded nut 312 can be threaded to fit against the lip 366 of the Luer Lok syringe 360.

In another contemplated embodiment, the recess 345 of the adapter 310 can be configured to engage threads on the tapered post 362 and/or nose 364 of the Luer Lok syringe 360. In such an embodiment, the threaded nut 312 can be configured to engage the threads 352 on the proximal end of the adapter 310 while cutting threads on the outer wall of the syringe 360.

In an alternate embodiment, the tapered post 360 of the Luer Lok syringe 360 can be removed to provide an enlarged opening to permit fluids, tissue, and cells to flow with less resistance.

It should be appreciated that the direct contact between the adapter 310 and the syringe 360 can provide the adapter 310 with the ability to better withstand the longitudinal forces and torque that are often applied during medical procedures without failure. That is, conventional adapters that do not have such a tight coupling to the syringe 360 can fail under lower amounts of longitudinal and rotational stresses than the break-away adapter 310.

For example, in lipoplasty procedures, differences in pressure gradients from harvesting the fat cells and injecting the fat cells can occur. That is, due to the viscus nature of fat, significant longitudinal forces must be applied to the composite apparatus of the syringe 360, adapter 310, and cannula 335 to inject fat into subcutaneous regions of the human body. This adapter 310 can be well suited to accommodate these longitudinal forces, which are known to overwhelm conventional adapters.

The diagrams in the Figures illustrate the architecture, functionality, and operation of possible implementations of systems and methods according to various embodiments of the present invention. It will also be noted that each block of the block diagrams and combinations of blocks in the block diagrams can be implemented by special purpose hardware-based systems that perform the specified functions or acts, or combinations of special purpose hardware and computer instructions.

What is claimed is:

1. A cannula adapter comprising:
an adapter body having an interior channel with opposing openings, wherein a first opening of the interior channel, located on a top surface of the adapter body, is sized and shaped to match a respective size and a respective shape for insertion of a cannula and a second opening of the interior channel is sized and shaped to match a respective size and a respective shape of a connection point of a medical device;
a breakpoint integrated throughout a horizontal cross-section of the adapter body, wherein, responsive to an application of transverse force to the adapter body that meets or exceeds a failure threshold of the breakpoint, the breakpoint separates the adapter body into two discrete sections, wherein separation of the adapter body decreases a likelihood of causing injury to a patient and the user of the medical device; and
a coupling mechanism that couples a bottom end of the adapter body to the connection point of the medical device, wherein engagement of the coupling mechanism forms an air-tight connection between the inserted cannula and the medical device that increases a failure threshold of the adapter body for withstanding longitudinal forces and torque applied by a user of the medical device during a medical procedure.

2. The cannula adapter of claim 1, wherein the breakpoint is longitudinally positioned where failures are known to have previously occurred under substantially similar conditions.

3. The cannula adapter of claim 1, further comprising:
a pressure release valve for mitigating pressure differentials between at least two of the cannula, the adapter body, and the medical device, wherein the pressure release valve is one of an integrated element of the adapter body and a separate element coupled to at least one of the adapter body and the coupling mechanism.

4. The cannula adapter of claim 1, wherein the coupling mechanism further comprises:
a plurality of threads integrated into an exterior surface of the adapter body proximate to the bottom end; and
a nut having grooves that correspond to the plurality of threads on the adapter body, wherein said nut is able to be screwed onto the adapter body and the connection point of the medical device, wherein the connection point of the medical device includes a matching plurality of threads.

5. The cannula adapter of claim 4, wherein the nut further comprises:
a set of cutting threads that physically cut thread grooves into the connection point of the medical device, wherein rotation of the nut simultaneously couples to the plurality of threads on the adapter body and cuts the thread grooves into the connection point of the medical device.

6. The cannula adapter of claim 1, wherein the coupling mechanism is a set of cutting threads embedded within the recess that physically cut thread grooves into the connection point of the medical device.

7. The cannula adapter of claim 1, wherein an exterior surface of the coupling mechanism is treated to reduce slippage during manual manipulation.

8. The cannula adapter of claim 1, wherein the adapter body and the coupling mechanism are made from a material suitable for medical use.

9. The cannula adapter of claim 1, wherein the medical device comprises one of a syringe, a wand attachment of a medical machine, and a handle for a tool attached to the medical machine.

10. A system comprising:
a cannula;
a syringe; and
an adapter that creates an air-tight connection between the cannula and the syringe, said adapter comprising:
an adapter body having an interior channel with opposing openings, wherein a first opening of the interior channel, located on a top surface of the adapter body, is sized and shaped to match a respective size and a respective shape for insertion of the cannula and a second opening of the interior channel is sized and shaped to match a respective size and a respective shape of a connection point of the syringe;
a breakpoint integrated throughout a horizontal cross-section of the adapter body, wherein, responsive to an application of transverse force to the adapter body that meets or exceeds a failure threshold of the breakpoint, the breakpoint separates the adapter body into two discrete sections, wherein separation of the adapter body decreases a likelihood of causing injury to a patient and the user of the medical device;
a coupling mechanism that couples a bottom end of the adapter body to the connection point of the syringe, wherein engagement of the coupling mechanism forms an air-tight connection between the inserted cannula and the syringe that increases a failure threshold of the adapter body for withstanding longitudinal forces and torque applied by a user of the syringe during a medical procedure.

11. The system of claim 10, wherein the syringe is a TOOMEY syringe and the connection point is a nose of the TOOMEY syringe.

12. The system of claim 10, wherein the syringe is a LUER LOK syringe and the connection point is a tapered post that protrudes from a nose of the LUER LOK syringe.

13. The system of claim 10, wherein the adapter further comprises:
a pressure release valve for mitigating pressure differentials between at least two of the cannula, the adapter body, and the syringe, wherein the pressure release valve is one of an integrated element of the adapter body and a separate element coupled to at least one of the adapter body and the coupling mechanism.

14. The system of claims 10, wherein the coupling mechanism further comprises:
a plurality of threads integrated into an exterior surface of the adapter body proximate to the bottom end; and
a nut having grooves that correspond to the plurality of threads on the adapter body, wherein said nut is able to be screwed onto the adapter body and the connection point of the medical device.

15. The system of claim 14, wherein the nut further comprises:
a set of cutting threads that physically cut thread grooves into the connection point of the syringe, wherein rotation of the nut simultaneously couples to the plurality of threads on the adapter body and cuts the thread grooves into the connection point of the syringe.

16. The system of claim 10, wherein the coupling mechanism is a set of cutting threads embedded within the adapter body that physically cut thread grooves into the connection point of the syringe.

17. A system comprising:
a cannula;
a manually-manipulated tool for a lipoplasty apparatus having a threaded connection point; and
an adapter that creates an air-tight connection between the cannula and the tool, said adapter comprising:
an adapter body having an interior channel with opposing openings, wherein a first opening of the interior channel, located on a top surface of the adapter body, is sized and shaped to match a respective size and a respective shape for insertion of the cannula and a second opening of the interior channel is sized and shaped to match a respective size and a respective shape of a connection point of the tool;
a breakpoint integrated throughout a horizontal cross-section of the adapter body, wherein, responsive to an application of transverse force to the adapter body that meets or exceeds a failure threshold of the breakpoint, the breakpoint separates the adapter body into two discrete sections, wherein separation of the adapter body decreases a likelihood of causing injury to a patient and the user of the lipoplasty apparatus; and
a coupling mechanism that couples a bottom end of the adapter body to the connection point of the tool, wherein engagement of the coupling mechanism forms an air-tight connection between the inserted cannula and the tool that increases a failure threshold of the adapter for withstanding longitudinal forces and torque applied by a user of the tool during a lipoplasty procedure.

18. The system of claim 17, wherein the adapter further comprises:
a pressure release valve for mitigating pressure differentials between at least two of the cannula, the adapter body, the tool, and the lipoplasty apparatus, wherein the pressure release valve is one of an integrated element of the adapter body and a separate element coupled to at least one of the adapter body and the coupling mechanism.

19. The system of claim 17, wherein the coupling mechanism further comprises:
a plurality of threads integrated into an exterior surface of the adapter body proximate to the bottom end; and
a nut having grooves that correspond to the plurality of threads on the adapter body and the connection point of the tool, wherein said nut is able to be screwed onto the adapter body and the connection point of the tool.

20. The system of claim 17, wherein the coupling mechanism is a nut that screws onto threads that are on exterior surfaces of the adapter body, proximate to the bottom end, and the tool.
